(19) United States
(12) Patent Application Publication
(10) Pub. No.: US 2013/0197183 A1
(43) Pub. Date: Aug. 1, 2013

(54) Process for Producing a Lactic Acid-Amine Complex

(71) Applicant: Edward Leslie Marshall, Imperial College (GB)

(72) Inventor: Edward Leslie Marshall, Imperial College (GB)

(73) Assignee: Plaxica Limited, London (GB)

(21) Appl. No.: 13/822,437

(22) PCT Filed: Oct. 17, 2011
(86) PCT No.: PCT/GB2011/001483

§ 371 Date: Apr. 15, 2013

(30) Foreign Application Priority Data
Oct. 18, 2010 (GB) 1017588.3

(51) Int. Cl. C07C 51/00

(52) U.S. Cl. CPC C07C 51/00
USPC 528/272; 562/515; 549/274

(57) ABSTRACT

A process for the production of a complex of lactic acid and either ammonia or an amine, comprising reacting one or more saccharides with barium hydroxide to produce a first reaction mixture comprising barium lactate, and contacting at least part of the first reaction mixture with ammonia or an amine and with carbon dioxide, or with the carbonate and/or bicarbonate salt of ammonia or an amine, to produce a second reaction mixture comprising said complex and barium carbonate.

The invention relates to a process for producing a lactic acid-amine complex.

Lactic acid is an important industrial chemical typically prepared from microbial fermentations of carbohydrates. A number of chemical processes for preparing lactic acid from carbohydrates are known. For example, GB 400,413, dating from 1933, describes an improved process for preparing lactic acid or lactates comprising reacting a carbohydrate-containing material with a strong alkali at a temperature of at least 200° C., preferably at a pressure of at least 20 atmospheres, and recovering the lactic acid so produced by adding sulfuric acid or zinc sulfate to the reaction mixture. GB 400,413 also states that it has been proposed to prepare lactic acid by treating certain sugars such as dextrose or sucrose with water and barium hydroxide at 160° C., and that prolonged contact of hexoses with dilute caustic soda, or treatment of pentoses with warm caustic potash, can also result in lactic acid. Such processes result in racemic lactic acid.

According to Boudrant et al, Process Biochem 40 (2005) p. 1642, “In 1987, the World production of lactic acid averaged approximately equal proportions being produced by chemical synthesis and fermentation processes”. Such chemical syntheses typically employed the hydrocyanation of acetaldehyde. However, chemical processes of this type have long been regarded as inefficient on an industrial scale, and today virtually all large scale production of the lactic acid available commercially is manufactured by fermentation processes, see for example Strategic Analysis of the Worldwide Market for Biorenewable Chemicals M2F2-39, Frost and Sullivan, 2009. In a typical fermentation process, biomass is fermented with microorganisms to produce either D- or L-lactic acid. Companies such as Cargill and Purac operate large-scale fermentation processes for the production of optically active lactic acid, and the patent literature is replete with improvements in such processes.

The product of a fermentation process is usually an optically active lactate salt, and recovery of lactic acid from such fermentation processes can be challenging. Many patent documents relate to lactic acid recovery, and a number rely on the preparation of a complex between lactic acid and an amine for the recovery. Such complexes can readily be converted into lactic acid or, if desired, used directly as feedstocks in processes for preparing derivatives of lactic acid. Thus, for example, U.S. Pat. No. 4,444,881 (Urbas, 1984) describes a process for the recovery of all organic acid (which may be lactic acid) from a fermentation reaction, which comprises converting the acid to its calcium salt, and adding a water-soluble tertiary amine carbonate (which may be prepared by addition of carbon dioxide to a solution or suspension of the tertiary amine in water). U.S. Pat. No. 5,510,526 (Baniel, 1994) claims a process, stated to be an improvement over that of Urbas, for the recovery of lactic acid from a lactate feed solution, comprising the use of an extractant comprising at least one water immiscible trialkylamine having a total of at least 18 carbon atoms in the presence of carbon dioxide at a partial pressure of at least 50 psig (about 3½ atmospheres, 3.4×10⁵ Pa).

A later document from inventor Baniel, U.S. Pat. No. 5,959,144 (1999) states that calcium lactate has been for many years, and still is, the primary fermentation produced material for the manufacture of lactic acid. It describes further research into the process of U.S. Pat. No. 5,510,526 and concludes that “the teachings of these publications [including U.S. Pat. No. 5,510,526] do not provide for a practical process for recovering lactic acid from calcium lactate”. An improvement is described in which carbohydrates such as dextrose or sucrose are added to the aqueous slurry of calcium lactate to increase the extractability of lactic acid by amine-based extractants.

An improved process has now been found which permits the economic production of complexes of lactic acid and either ammonia or an amine, without the need to use additives such as carbohydrates, as advocated by Baniel.

Accordingly, the invention provides a process for the production of a complex of lactic acid and either ammonia or an amine (“the Complex”), comprising reacting one or more saccharides with barium hydroxide to produce a first reaction mixture comprising barium lactate, and contacting at least part of the first reaction mixture with ammonia or an amine and with carbon dioxide, or with the carbonate and/or bicarbonate salt of ammonia or an amine, to produce a second reaction mixture comprising the Complex and barium carbonate.

In the process of the invention, one or more saccharides is reacted with barium hydroxide. The saccharide may be a mono-, di-, tri- or poly-saccharide, with disaccharides and, especially, monosaccharides, being preferred. Suitable disaccharides include sucrose, lactose, lactulose, maltose, trehalose and cellobiose. Suitable monosaccharides include for example hexose monosaccharides, for example glucose, fructose, psicose and mannose. Pentoses may also be used, for example arabinose, xylose, ribose, xylulose and ribulose. In one embodiment, the saccharide comprises glucose. In another embodiment, the saccharide comprises fructose. Suitable monosaccharides also include pentose monosaccharides, for example arabinose. Mixtures of saccharides may be used. For example, the saccharide may comprise a mixture of two or more monosaccharides, for example a mixture of glucose and fructose.

Monosaccharides may be obtained from any known monosaccharide source, for example a higher saccharide such as sucrose, starch or cellulose. By way of example, a mixture of glucose and fructose (known as invert sugar) may be obtained from sucrose by enzymatic hydrolysis using a sucrase or invertase, or by heating an aqueous solution of the disaccharide in the presence of an acidic catalyst such as sulfuric acid, citric acid or ascorbic acid. Alternatively, glucose may be obtained by enzymatic hydrolysis (e.g. using an amylase) of starch contained in biomass feedstocks, for example maize, rice or potatoes. Where a saccharide other than a monosaccharide is used as a starting material in the process of the invention and reacted with barium hydroxide, it is possible that monosaccharide is generated in situ and subsequently reacts with barium hydroxide.

The process of the invention is typically carried out in the presence of one or more solvents. In particular, the reaction between the saccharide and barium hydroxide is normally carried out in the presence of water. Some commercial sources of saccharide, particularly sources of monosaccharide and disaccharide, contain water, and such feedstocks may readily be used in the process of the invention. In certain embodiments, the reaction between the saccharide and barium hydroxide may take place in the presence of additional water (i.e. additional to that present in the starting materials). The reaction between the saccharide and barium hydroxide may also, if desired, take place in the presence of one or more organic solvents, for example an oxygenate such as an alcohol, ester, ether, or ketone; and/or in the presence of one or more reactive extractants such as an amine. However, in a preferred embodiment, the reaction between the saccharide and barium hydroxide does not take place in the presence of an organic solvent.

Barium hydroxide reacts with saccharide to produce barium lactate. Sources of barium hydroxide such as barium oxide may be used in the process of the invention, barium oxide being converted into barium hydroxide in the presence of water. The barium hydroxide generated in situ reacts with the saccharide to produce barium lactate.

The ratio of barium hydroxide to saccharide should be sufficient to effect high conversion of saccharide to barium lactate. For example, when the saccharide comprises glucose, for each mole of glucose there is preferably used at least one mole of barium hydroxide (i.e. the molar ratio of barium hydroxide to saccharide (calculated as monosaccharide) is at least 1:1). Excess quantities of barium hydroxide may be used, for example the molar ratio of barium hydroxide to saccharide (calculated as monosaccharide) may be up to 10:1. In a preferred embodiment, the molar ratio of barium hydroxide to saccharide (calculated as monosaccharide) is from 1:1 to 5:1, more preferably 1.2:1 to 4:1, especially 1.2:1 to 2:1. The present invention also encompasses molar ratios of barium hydroxide to saccharide (calculated as monosaccharide) that are lower than 1:1, although this is not preferred since use of sub-stoichiometric quantities of barium hydroxide will generally lead to lower conversion of saccharide to barium lactate.

The conversion of saccharide to barium lactate may be carried out at room temperature, although the reaction is preferably carried out at elevated temperature, for example at a temperature of up to 150° C. Preferably, saccharide is reacted with barium hydroxide at a temperature of from 50° to 120° C., more preferably from 70° to 110° C., for example from 75 to 100° C. In one embodiment, saccharide is reacted with barium hydroxide at 80° C. In another embodiment, saccharide is reacted with barium hydroxide in water at reflux.

In a preferred embodiment, an aqueous solution of at least one saccharide, especially a monosaccharide, is added over a period of time to a mixture of barium hydroxide and water that is at elevated temperature, for example at reflux. Slow addition of the saccharide generally leads to a reduction in the formation of side products during the process of the invention, and leads to an improved conversion of saccharide into barium lactate. Preferably the aqueous solution of saccharide is added over a period of at least 30 minutes, more preferably over at least 1 hour, most preferably over at least 2 hours.

The aqueous solution of at least one saccharide preferably has a concentration of less than 4.0 M, more preferably 0.2-2.0 M, most preferably 0.5-1.5 M.

The reaction of saccharide with barium hydroxide produces a first reaction mixture comprising barium lactate. The process leads to the production of racemic barium lactate.

At least a portion of the first reaction mixture is contacted with ammonia or an amine and with carbon dioxide, or with the carbonate and/or bicarbonate salt of ammonia or an amine, to produce a second reaction mixture comprising the Complex and barium carbonate. It is believed that when the ammonia or amine and carbon dioxide are added to the first reaction mixture, the corresponding carbonate and/or bicarbonate salt of the ammonia or amine (i.e. the ammonium carbonate or bicarbonate) is produced in situ, and that the carbonate and/or bicarbonate salt of ammonia or the amine reacts with barium lactate to produce the Complex and barium carbonate.

Carbon dioxide may be added in any suitable form, typically as a solid or, preferably, as a gas. Depending on the reactants used in the process of the invention, the step of contacting at least part of the first reaction mixture with ammonia or an amine and with carbon dioxide may be carried out at substantially atmospheric pressure or at moderate pressure, for example 1 to 1.5 atmospheres. If a pressure vessel is used, higher partial pressures of carbon dioxide may be used.

The amines used in the process of the invention include primary, secondary and tertiary amines, of which tertiary amines are preferred. The amines used in the process of the invention are preferably alkylamines, most preferably trialkylamines. Examples of suitable trialkylamines include triethylamine, tripropylamine, tributylamine, tripentylamine and trihexylamine. The amine used may be a single component, or it may be a mixture of amines. Suitably an equivalent amount or an excess of ammonia or amine based on the lactic acid is used. For example, at least one equivalent, up to 10 equivalents, preferably up to 8, more preferably up to 6, still more preferably up to 4, especially 2 equivalents, of ammonia or amine, may be used.

In one aspect, ammonia or an amine that is at least partially water soluble is used. Amines that are at least partially water soluble permit the use of carbon dioxide at low or atmospheric reaction pressures (e.g. by bubbling a slight overpressure of carbon dioxide gas from a pressurised cylinder or other carbon dioxide source into a reaction mixture that is substantially at atmospheric pressure). As defined herein, an amine that is at least partially water soluble has a solubility in water of at least 1 g per litre at 25° C. Preferably, the amine is an alkylamine that has less than 12 carbon atoms. In one embodiment, the amine has less than 10 carbon atoms. In another embodiment, the amine has less than 9 carbon atoms. Examples of suitable amines include t-butylamine, octylamine, diethylamine, diisopropylamine and triethylamine. In one embodiment, the amine is triethylamine.

In another aspect of the invention, the amine is an amine that is immiscible with water. Such amines generally have a total of at least 12 carbon atoms. In one embodiment, the amine has at least 18 carbon atoms. In another embodiment the amine has at least 24 carbon atoms. The amine that is immiscible with water preferably has up to 42 carbon atoms, for example the amine may have from 12 to 42 carbon atoms, from 18 to 42 carbon atoms, or from 24 to 42 carbon atoms. Examples of such amines include trihexylamine, triheptylamine, trioctylamine (e.g. tri-(n-octyl) amine, triisooctylamine, tri-(2-ethylhexyl)amine), trioctylamine, tridecylamine, tridodecylamine and Alamine 336™. The use of amines that are immiscible with water facilitates the separation of the Complex from the second reaction mixture, by partitioning of the Complex into the amine phase of a biphasic water-amine mixture. In that case, the step of contacting at least part of the first reaction mixture with an amine and with carbon dioxide is preferably carried out using carbon dioxide at higher reaction pressures so that good conversion of barium lactate into Complex and barium carbonate is achieved. Preferably the carbon dioxide in the reaction vessel is maintained at a partial pressure of at least 3 atm (3×10⁵ Pa), more preferably at least 5 atm (5×10⁵ Pa) and most preferably from 10-20 atm (1×10⁶-2×10⁶ Pa).

As an alternative to contacting the first reaction mixture containing barium lactate with an amine and carbon dioxide, the reaction mixture may instead be contacted with the carbonate or bicarbonate salt of ammonia or an amine. For example, an alkylammonium carbonate or bicarbonate such as triethylammonium bicarbonate may be added to the first reaction mixture. The carbonate or bicarbonate salt of ammonia or the amine may be added neat, or alternatively the carbonate or bicarbonate salt of ammonia or the amine may be added as a solution. Suitable solvents include water and aqueous/organic mixtures, for example water/amine mixtures.

The carbonate or bicarbonate salt of ammonia or an amine is preferably prepared from ammonia or an amine and carbon dioxide. For example, it may be produced from the addition of carbon dioxide to a solution of ammonia or an amine in water. The resulting solution containing the carbonate or bicarbonate salt of ammonia or the amine may then be contacted with the first reaction mixture comprising barium lactate.

The product formed by contacting the first reaction mixture comprising barium lactate with ammonia or an amine and with carbon dioxide is referred to herein as a Complex. In such a Complex, both ion pair and hydrogen bond interactions may occur between the racemic lactic acid and ammonia or the amine. The precise form of the Complex will depend on the environment in which it is found. The Complex may be regarded as a partly ionised liquid or, alternatively, as a simple salt between the acid and ammonia or the amine, existing in equilibrium with free acid and ammonia or amine. For example, in the case of tri(n-octyl)amine, trioctylammonium lactate may be produced.

The process of the invention produces a second reaction mixture comprising the Complex and barium carbonate. Since the Complex is produced from racemic barium lactate, the Complex is expected to be racemic.

In one aspect, the Complex may be separated from the second reaction mixture by partitioning of the Complex into the amine-rich phase of a biphasic mixture comprising water and amine. As described above, when an amine is used that is immiscible with water, a biphasic water-amine mixture results. Partitioning of the Complex into the amine layer facilitates the separation of the Complex from the second reaction mixture. In order to aid the extraction of the Complex into the amine-rich phase, one or more organic solvents may also be added. Examples of suitable solvents are described in U.S. Pat. No. 5,510,526 (Baniel, 1994).

When the amine is at least partially water soluble, if the Complex is to be extracted from the second reaction mixture, it will usually be necessary to add at least one organic solvent in order to form a biphasic mixture. Examples of suitable solvents are described in U.S. Pat. No. 4,444,881 (Urbas, 1984).

The process of the invention also produces barium carbonate which typically precipitates from the second reaction mixture. Other barium salts such as barium bicarbonate may also be produced. The precipitation of insoluble barium carbonate from the second reaction mixture assists in driving the conversion of barium lactate to Complex. In one embodiment, barium carbonate is separated from the second reaction mixture by filtration. Preferably, barium carbonate is separated from the second reaction mixture and is then converted into barium oxide, typically by conventional calcination technology. The barium oxide may then be recycled to the process of the invention, being added to an aqueous solution containing at least one saccharide, generating barium hydroxide in situ. Alternatively, the barium oxide may be converted to barium hydroxide in a separate step, the barium hydroxide then being used in the process of the invention.

The Complex may be subject to purification and/or additional processing steps. For example, where organic solvent is present in the extract containing the Complex, the organic solvent may be removed by distillation and optionally recycled. In the case where the organic solvent is an alcohol, the corresponding lactate ester may be produced and separated by distillation, see for example U.S. Pat. No. 5,453, 365 (Sterzel et al, 1995).

The process of the present invention may be carried out in a batch, semi-continuous or continuous process.

The process of the invention may be carried out under ambient or inert atmosphere. For example, the process may be carried out using equipment that is open to the air, or may be carried out under a nitrogen or argon atmosphere.

The Complex may be converted into lactic acid, and the present invention further provides a process for the preparation of lactic acid, which comprises producing a Complex by a process according to the invention, and converting the Complex into lactic acid. For example, following separation of an amine-rich phase containing the Complex from the second reaction mixture, lactic acid may be obtained from the amine-rich phase by distillation.

The Complex may also be reacted to form lactide, a cyclic dimer of lactic acid that is itself useful in the production of polylactic acid. The invention therefore further provides a process for the production of lactide, comprising producing a Complex by a process according to the invention, optionally converting the Complex into lactic acid, and converting the Complex or lactic acid into lactide. For example, the Complex may be heated to produce a pre-polymer or oligomer of lactic acid which is contacted with a transesterification catalyst to produce lactide. Instead of using the Complex, lactic acid may instead be reacted to form lactide. As described above for the Complex, lactic acid may also be heated to produce a pre-polymer or oligomer of lactic acid which is contacted with a transesterification catalyst to produce lactide.

There are three forms of lactide, (S,S)- or L-lactide, (R,R)- or D-lactide, and (R,S)- or meso-lactide. Racemic and meso-lactide may be separated by standard separation techniques, for example by distillation, solvent extraction, or crystallisation.

Lactide, and particularly racemic lactide, may be polymerized to form polylactic acid. The invention therefore further provides a process for the production of polylactic acid, comprising producing lactide by a process according to the invention, and polymerising the lactide to form polylactic acid. This polymerisation may be carried out by contacting lactide with a catalyst at elevated temperature.

The present invention provides a high-yielding, economic process for the preparation of a complex of lactic acid with either ammonia or an amine from readily available starting materials. It steps away from long-established norms of lactic acid manufacture and, surprisingly, provides a chemical process comparing very favourably on economic terms with fermentation processes. Further, using barium, it demonstrates a most surprising improvement in yield of the Complex compared with the corresponding process based on the use of calcium, as illustrated in the Examples which follow. Given the prior art, the skilled man would have expected that the use of calcium in a process of this type would provide optimal technical results. The improved results obtained appear to be unique to the use of barium, and could not have been predicted.

The following Examples illustrate the invention.

### EXAMPLE 1

Step 1. 1.52 g of Ba(OH)₂.H₂O (8 mmol, 2 molar equivalents) were added to 20 mL of deionised water in a 100 mL two-necked round bottom flask fitted with a Teflon magnetic stirrer and a condenser. The suspension was heated to an internal temperature of 80° C. (monitored with a thermocouple placed inside the reaction mixture) and 20 mL of 0.2 mol/L glucose solution (4 mmol, 1 molar equivalent) was added in a dropwise manner using a syringe pump over 4 hours (flow rate of 5 mL/h). Following the addition of the glucose solution the mixture was left to cool to room temperature with continuous stirring.

A 3 mL sample of the resulting reaction mixture was added to approximately 1 mL Amberlite™ 120 hydrogen form cation exchange resin and stirred for 30 minutes, prior to HPLC analysis utilising a Perkin Elmer 200 series HPLC fitted with an RI detector, ICE ION-300 ion exclusion column and Brownlee polypore-H guard column. Samples were run at a flow rate of 0.3 mL/h, with a detector temperature 40° C., column oven temperature of 50° C. and a 0.005 mol/L in H₂SO₄ mobile phase leading to a 480 PSI system pressure. The concentration of lactic acid in the reaction mixture was quantified by reference to a standard calibration curve. A lactate yield of 46.6% was obtained (two repeats, SEM±1.0).

Step 2. 1.5 mL of triethylamine (6 molar equivalents, calculated by reference to the preceding quantification of barium lactate) were added to the reaction mixture and stirred for a few minutes at room temperature, prior to a flow of excess gaseous CO₂ (ref 150102-V, BOC) being bubbled through the mixture for 60 minutes. The resulting mixture was filtered under gravity using Whatman filter paper (pore size 11 μm) and the filter cake washed with deionised water. The cake was then dried (room temperature, 24 h; then 60° C., 48 h) to remove any remaining water and amine, and the resulting dry mass of barium carbonate was weighed. A 95.7% yield of barium carbonate over Step 2 was obtained (two repeats, SEM±0.4), corresponding to an almost quantitative separation of lactic acid-triethylamine complex.

### Example 2

### Comparative Example Using Calcium Hydroxide

Example 1 was repeated, but using 0.593 g Ca(OH)₂ (8 mmol, 2 molar equivalents) instead of Ba(OH)₂. In step 1, a lactate yield of 37.5% was obtained (two repeats, SEM±1.0). In step 2, a carbonate yield of 99.0% was obtained (two repeats, SEM±7.2). Thus the use of barium hydroxide rather than calcium hydroxide results in an increase of 24% in the amount of lactate obtained in the first step, and lactic acid-triethylamine complex separated in the second step.

The results clearly demonstrate a significant and unexpected improvement in the yield of metal lactate from glucose when using barium hydroxide instead of calcium hydroxide, the use of barium hydroxide generating 24% more lactate than the use of calcium hydroxide.

### EXAMPLE 3

Step 1. 1.51 g of Ba(OH)₂.H₂O (8 mmol, 2 molar equivalents) were added to 20 mL of deionised water in a 100 mL three-necked round bottom flask fitted with a Teflon magnetic stirrer and a condenser. The suspension was heated to an internal temperature of 80° C. (monitored with a thermocouple placed inside the reaction mixture) and 20 mL of 0.2 mol/L glucose solution (4 mmol, 1 molar equivalent) was added in a dropwise manner using a syringe pump over 4 hours (flow rate of 5 mL/h). Following the addition of the glucose solution the mixture was left to cool to room temperature with continuous stirring.

A 0.5 mL sample of the resulting reaction mixture was added to 4.5 mL HPLC grade water and approximately 0.5 g Amberlite™ 120 hydrogen form cation exchange resin and stirred for 5 minutes, prior to HPLC analysis utilising a Perkin Elmer 200 series HPLC fitted with an RI detector, ICsep ICE-ION-300 ion exclusion column and Brownlee polypore-H guard column. Samples were run at a flow rate of 0.3 mL/h, with a detector temperature of 40° C., column oven temperature of 50° C. and a 0.005 mol/L H₂SO₄ mobile phase leading to a 530PSI system pressure. The concentration of lactic acid in the reaction mixture was quantified by reference to a standard calibration curve. A lactate yield of 48.6% was obtained.

Step 2. 1.6 mL ammonium hydroxide solution (28% w/w, 11.52 mmols, 6 molar equivalents, calculated by reference to the preceding quantification of barium lactate) were added to the reaction mixture from Step 1 and stirred for a few minutes at room temperature, prior to a flow of excess gaseous CO₂ (ref 150102-V, BOC) being bubbled through the mixture for 60 minutes. The resulting mixture was filtered under gravity using Whatman filter paper (pore size 11 μm) and the filter cake washed with deionised water. The cake was then dried (room temperature, 3 h; then 60° C., 24 h) to remove any remaining water and amine, and the resulting dry mass of barium carbonate was weighed. A 98.7% yield of barium carbonate over Step 2 was obtained, corresponding to an almost quantitative separation of lactic acid-triethylamine complex. HPLC analysis (per Step 1) showed that >96% of the lactate species was retained in solution.

### EXAMPLE 4

132 g barium hydroxide octahydrate (0.42 mol) were dissolved in 350 mL demineralised water in a 1 L glass flanged flask and heated to 95° C. 83 g of 1 M invert sugar solution, diluted with a further 350 mL water, was then added dropwise over 3 h. HPLC analysis revealed a lactate yield of 57.8%.

After cooling to room temperature, 200 g of the product mixture liquors were then transferred to a 1 L round bottomed flask fitted with an overhead stirrer, a water-cooled condenser and a dropping funnel. Ammonium carbonate (10.6 g, ca. 1.1 equivalents relative to Ba(OH)₂.8H₂O) was dissolved in 50 mL demineralised water and added dropwise at ambient temperature over 30 minutes. The dark brown appearance of the starting liquors quickly became a pale brown and large quantities of a pale coloured precipitate formed. At the end of the addition the reaction mixture was centrifuged and the recovered precipitate washed with 100 mL water and centrifuged a second time. Analysis of the combined decanted fractions showed a 92% recovery of the lactate anions as ammonium lactate.

1. A process for the production of a complex of lactic acid and either ammonia or an amine, comprising reacting one or more saccharides with barium hydroxide to produce a first reaction mixture comprising barium lactate, and contacting at least part of the first reaction mixture with ammonia or an amine and with carbon dioxide, or with the carbonate and/or bicarbonate salt of ammonia or an amine, to produce a second reaction mixture comprising said complex and barium carbonate.

2. A process as claimed in claim 1, wherein said saccharide is a monosaccharide.

3. A process as claimed in claim 2, wherein said monosaccharide comprises glucose and/or fructose.

4. A process as claimed in claim 3, wherein said monosaccharide comprises a mixture of glucose and fructose.

5. A process as claimed in claim 1, wherein said saccharide is reacted with barium hydroxide at a temperature of from 50 to 120° C.

6. A process as claimed in claim 5, wherein said saccharide is reacted with barium hydroxide at a temperature of from 70 to 110° C.

7. A process as claimed in claim 1, wherein the barium hydroxide has been produced in situ from barium oxide and water.

8. A process as claimed in claim 1, wherein the molar ratio of barium hydroxide to saccharide, calculated as monosaccharide, is from 1:1 to 5:1.

9. A process as claimed in claim 1, wherein barium carbonate is separated from the second reaction mixture by filtration.

10. A process as claimed in claim 1, wherein said complex is separated from said second reaction mixture by partitioning of said complex into the amine-rich phase of a biphasic mixture comprising water and amine.

11. A process as claimed in claim 10, wherein the amine-rich phase comprises at least one organic solvent.

12. A process as claimed in claim 1, wherein at least part of the first reaction mixture is contacted with ammonia or an amine and with carbon dioxide.

13. A process as claimed in claim 1, wherein at least part of the first reaction mixture is contacted with the carbonate and/or bicarbonate salt of ammonia or an amine.

14. A process as claimed in claim 13, wherein the carbonate and/or bicarbonate salt of ammonia or an amine is produced from ammonia or an amine and carbon dioxide.

15. A process as claimed in claim 1, wherein the amine is an alkylamine having less than 12 carbon atoms.

16. A process as claimed in claim 1, wherein the amine is an alkylamine that is at least partially water soluble.

17. A process as claimed in claim 1, wherein the amine is a trialkylamine.

18. A process as claimed in claim 15, wherein the amine is triethylamine.

19. A process as claimed in claim 1, the amine is immiscible with water.

20. A process as claimed in claim 1, wherein the amine has at least 18 carbon atoms.

21. A process as claimed in claim 19, wherein the amine is selected from the group consisting of trihexylamine, trioctylamine and Alamine 336™.

22. A process as claimed in claim 1, comprising separating barium carbonate from the second reaction mixture, converting the barium carbonate to barium oxide, converting the barium oxide to barium hydroxide, and recycling to the process.

23. A process for the production of lactic acid, comprising producing a complex of lactic acid and either ammonia or an amine by a process according to claim 1, and converting said complex into lactic acid.

24. A process for the production of lactide, comprising producing a complex of lactic acid and either ammonia or an amine by a process according to claim 1, and converting said complex into lactide.

25. A process for the production of polylactic acid, comprising producing lactide by a process according to claim 24, and polymerising the lactide to form polylactic acid.

26. A process for the production of lactide, comprising producing lactic acid by a process according to claim 23, and converting said lactic acid into lactide.

27. A process for the productions of polylactic acid, comprising producing lactide by a process according to claim 26, and polymerising the lactide to form polylactic acid.
